(19) United States
(12) Patent Application Publication
(10) Pub. No.: US 2023/0104596 A1
(43) Pub. Date: Apr. 6, 2023

(54) BIOLOGICAL INFORMATION MEASURING GARMENT FOR CATTLE

(71) Applicants: TOYOBO CO., LTD., Osaka (JP); TOYOBO STC CO., LTD., Osaka (JP)

(72) Inventors: Yoko KOMATSU, Shiga (JP); Yuichiro OMOTE, Osaka (JP); Tomoyuki MIYAMOTO, Osaka (JP)

(73) Assignees: TOYOBO CO., LTD., Osaka (JP); TOYOBO STC CO., LTD., Osaka (JP)

(21) Appl. No.: 17/795,377

(22) PCT Filed: Jan. 19, 2021
(86) PCT No.: PCT/JP2021/001610

§ 371 Date: Jul. 26, 2022

(30) Foreign Application Priority Data
Jan. 28, 2020 (JP) 2020-011435

(51) Int. Cl. A61B 5/25; A61B 5/00

(52) U.S. Cl. CPC A61B 5/25; A61B 5/6804; A41D 13/1281

(57) ABSTRACT

An object of the present invention is to provide a biological information measuring garment for cattle that allows easy measurement of biological information. The biological information measuring garment for cattle includes a clothing fabric; and an electrode provided on a skin-side surface of the clothing fabric, the clothing fabric including a first band portion on one end and a second band portion on another end in a body peripheral direction of the clothing fabric, and the clothing fabric having a larger area in a front-side region ahead of a region from the first band portion to the second band portion of the clothing fabric than an area in a back-side region behind the region from the first band portion to the second band portion.

### TECHNICAL FIELD

The present invention relates to a biological information measuring garment for cattle.

### BACKGROUND ART

In recent years, biological information measuring garments are drawing attention in a health monitoring field, a medical field, a medical treatment and education field, and a rehabilitation field. The inventors of the present invention have, in Patent Document 1, identified a measurement position that allows stable measurement of biological information and proposed a sensing wear to which a highly close-attachment flexible electrode is mounted.

### RELATED ART DOCUMENT

### Patent Document

Patent Document 1: JP-A-2017-29692

### SUMMARY OF THE INVENTION

### Problem to be Solved by the Invention

As seen in Patent Document 1, biological information measuring garments worn by human beings are being variously developed. In the livestock and dairy industry, however, a biological information measuring garment for cattle has not been developed yet, despite the fact that there is a desire of making cattle wear a biological information measuring garment to perceive the health of the cattle. The present invention has been made under the circumstances, and an object of the present invention is to provide a biological information measuring garment for cattle that allows easy measurement of biological information.

### Solutions to the Problem

A biological information measuring garment for cattle according to the present invention, which can solve the problem is as per [1] below.

A biological information measuring garment for cattle, the biological information measuring garment including:
a clothing fabric; and
an electrode provided on a skin-side surface of the clothing fabric,
the clothing fabric including a first band portion on one end and a second band portion on another end in a body peripheral direction of the clothing fabric, and
the clothing fabric having a larger area in a front-side region ahead of a region from the first band portion to the second band portion of the clothing fabric than an area in a back-side region behind the region from the first band portion to the second band portion.

The clothing fabric having a larger area in a front-side region than an area in a back-side region as in [1] above makes the electrode less likely to be positionally displaced and therefore improves the measurement accuracy of biological information. The biological information measuring garment for cattle according to the present invention preferably includes any configuration of [2] to [11] below.

The biological information measuring garment according to above [1], wherein

the electrode is provided in the region from the first band portion to the second band portion of the clothing fabric.

The biological information measuring garment according to above [1] or [2], wherein

the clothing fabric includes a portion having the electrode provided therein, the portion having a length in a front-back direction that is shorter than a maximum length in the front-back direction of the clothing fabric.

The biological information measuring garment according to any one of above [1] to [3], wherein,

the first band portion has a length in the front-back direction that is shorter than the length in the front-back direction of the portion having the electrode provided therein.

The biological information measuring garment according to any one of above [1] to [4], wherein,

the second band portion has a length in the front-back direction that is shorter than the length in the front-back direction of the portion having the electrode provided therein.

The biological information measuring garment according to any one of above [1] to [5], wherein

the front-side region of the clothing fabric includes a curved portion including an outer edge curving toward a back direction.

The biological information measuring garment according to any one of above [1] to [6], wherein

the electrode includes an insulating layer formed on the skin-side surface of the clothing fabric, and a conductive layer formed on the insulating layer.

The biological information measuring garment according to any one of above [1] to [7], wherein

the conductive layer includes a conductive filler and a resin.

The biological information measuring garment according to any one of above [1] to [8], wherein

the electrode is formed of a conductive structure, and when the conductive structure has a load of 14.7 N applied thereto in a body length direction or a body width direction, the conductive structure has an extension percentage of 3% or more and 60% or less in at least one of the directions.

The biological information measuring garment according to any one of above [1] to [9], being configured to measure electrocardiographic information.

The biological information measuring garment according to any one of above [1] to [10], being configured to cover at least a part of a thorax, an abdomen, a back, a front leg, a rear leg, a neck, or a face.

### Effects of the Invention

According to the present invention, the configurations described above enable provision of a biological information measuring garment for cattle that allows easy measurement of biological information.

### BRIEF DESCRIPTION OF THE DRAWINGS

FIG. 1 is a plan view of a outer side of a biological information measuring garment for cattle according to an embodiment of the present invention.

FIG. 2 is a view of the biological information measuring garment for cattle according to the embodiment of the present invention that is worn by a cow.

### MODE FOR CARRYING OUT THE INVENTION

A biological information measuring garment for cattle according to the present invention includes a clothing fabric and an electrode provided on a skin-side surface of the clothing fabric, the clothing fabric including a first band portion on one end and a second band portion on another end in a body peripheral direction of the clothing fabric, and the clothing fabric having a larger area in a front-side region ahead of a region from the first band portion to the second band portion of the clothing fabric than an area in a back-side region behind the region from the first band portion to the second band portion. The clothing fabric having a larger area in a front-side region than an area in a back-side region makes the electrode less likely to be positionally displaced and therefore improves the measurement accuracy of biological information.

Hereinafter, the present invention is more specifically described on the basis of the following embodiment. The present invention, however, is not limited by the following embodiment, and can also be absolutely implemented with appropriate changes to the embodiment within a scope in compliance with the intent described above and below, and all the changes are to be encompassed within the technical scope of the present invention. In each of the drawings, the symbols of members, or the like are sometimes omitted for convenience. In such a case, the description and other drawings are to be referred to. Further, the drawings put priority on contributing to understanding of the features of the present invention, and therefore the dimensions of various members in the drawings are sometimes different from the actual dimensions thereof.

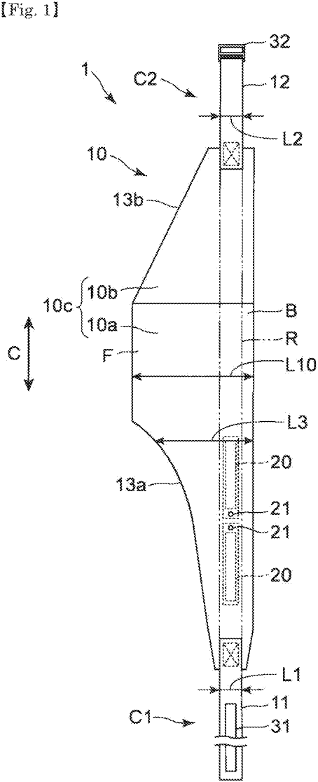

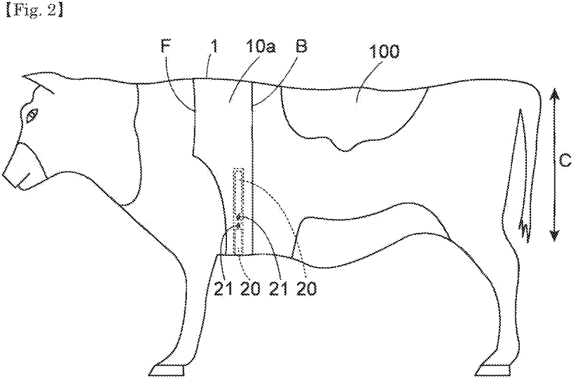

With reference to FIGS. 1 and 2, a biological information measuring garment for cattle according to an embodiment of the present invention is described. FIG. 1 is a plan view of a outer side opposite to a skin side of a biological information measuring garment for cattle according to an embodiment of the present invention. FIG. 2 is a view of the biological information measuring garment for cattle according to the embodiment of the present invention that is worn by a cow. In FIGS. 1 and 2, the dashed lines are lines representing members provided on the skin side. On the other hand, the dashed-two dotted line is a virtual line representing a central region R described later.

As illustrated in FIG. 1, a biological information measuring garment for cattle 1 includes a clothing fabric 10 and an electrode 20 provided on a skin-side surface of the clothing fabric 10. Further, the clothing fabric 10 includes a first band portion 11 on one end C1 and a second band portion 12 on another end C2 in a body peripheral direction C of the clothing fabric 10. The clothing fabric 10 has a larger area in a front-side region F ahead of a region (hereinafter sometimes called a central region R) from the first band portion 11 to the second band portion 12 of the clothing fabric 10 than an area in a back-side region B behind the central region R of the clothing fabric 10. The clothing fabric 10 having a larger area in the front-side region F than an area in the back-side region B of the clothing fabric 10 makes the electrode 20 less likely to be positionally displaced and therefore improves the measurement accuracy of biological information, for example, when the biological information measuring garment for cattle 1 is worn by a cow 100 as illustrated in FIG. 2. In FIG. 2, the front side means a head side of the cow 100, and the back side means a tail side of the cow 100.

The area (cm²) of the front-side region F of the clothing fabric 10 should be larger than the area (cm²) of the back-side region B in a plan view. Specifically, the area of the front-side region F is preferably 1.1 times or more, more preferably 1.5 times or more, further preferably 2.0 times or more the area of the back-side region B. On the other hand, the upper limit is not particularly limited, and may be 20 times or less, 15 times or less, 10 times or less, or 7 times or less. The clothing fabric 10 should include at least the front-side region F, but does not need to include the back-side region B. That is, the area of the back-side region B may be 0 (cm²). In this case, the area (cm²) of the front-side region F is to be larger than the area 0 (cm²) of the back-side region B.

In FIG. 1, the clothing fabric 10 includes a main portion 10c, the first band portion 11, and the second band portion 12. The main portion 10c is longer in a front-back direction than the first band portion 11 and the second band portion 12. The main portion 10c is not particularly limited in terms of the shape as long as it has a larger area in the front-side region F than an area in the back-side region B. For example, the main portion 10c may have a first tapered portion 13a whose length in the front-back direction is shorter toward the first band portion 11 as illustrated in FIG. 1. The main portion 10c may also have a second tapered portion 13b whose length in the front-back direction is shorter toward the second band portion 12 as illustrated in FIG. 1. These configurations enable easy fastening of the biological information measuring garment for cattle 1. Examples of the shape of the first tapered portion 13a and the second tapered portion 13b include a curve shape including an outer edge curving toward a back direction, and a so-called linearly tapered shape tapered at a constant angle. Alternatively, the main portion 10c may have a rectangle such as a square or an oblong.

The front-side region F of the clothing fabric 10 preferably includes a curved portion including an outer edge curving toward a back direction. This configuration enables easy fastening of the biological information measuring garment for cattle 1. In FIG. 1, the first tapered portion 13a corresponds to the curved portion.

In FIG. 1, the main portion 10c includes a first clothing fabric 10a and a second clothing fabric 10b. Using clothing fabrics made of different materials as the first clothing fabric 10a and the second clothing fabric 10b enables impartation of various functions to the biological information measuring garment for cattle 1. The first clothing fabric 10a and the second clothing fabric 10b, however, are not limited to the use of clothing fabrics made of different materials, but may be clothing fabrics made of the same material. In addition, the first clothing fabric 10a and the second clothing fabric 10b may be seamlessly and integrally formed. Further, the main portion 10c is not limited to being formed of the first clothing fabric 10a and the second clothing fabric 10b, but may be formed using three or more clothing fabrics.

Specific examples of the first clothing fabric 10a and the second clothing fabric 10b include a knitted fabric, a woven fabric, and a non-woven fabric. Among these fabrics, a knitted fabric is preferable because it has excellent stretchability. Examples of the knitted fabric include a weft knitted fabric and a warp knitted fabric. Among these fabrics, a warp knitted fabric is preferable. The weft knitted fabric includes a circular knitted fabric. Examples of the weft knitted fabric (circular knitted fabric) include fabrics with stitches such as jersey stitch (plain stitch), bare jersey stitch, welt jersey stitch, fraise stitch (rib stitch), purl stitch, half tubular stitch, interlock stitch, tuck stitch, float stich, half cardigan stitch, lace stitch, and plating stitch. Examples of the warp knitted fabric include fabrics with stitches such as single denbigh stitch, open-loop denbigh stitch, single atlas stitch, double cord stitch, half stitch, half base stitch, satin stitch, single tricot stitch, double tricot stitch, half tricot stitch, single raschel stitch, double raschel stitch, and jacquard stitch. Examples of the woven fabric include woven fabrics formed by plain weave, twill weave, and satin weave. The woven fabric is not limited to a single woven fabric, but may be a multiple woven fabric such as a double woven fabric or a triple woven fabric. These knitted fabrics and woven fabrics, and the like may be formed in a mesh pattern.

As the first clothing fabric 10a and the second clothing fabric 10b can also be used one obtained by sewing these knitted fabrics, woven fabrics, non-woven fabrics, or the like to form a bag, putting, for example, a foamed sheet such as a urethane sponge sheet, or inner cotton in an internal space of the bag, and sealing the bag.

The first clothing fabric 10a preferably has a larger thickness than the thickness of the second clothing fabric 10b. This configuration makes it easy for the first clothing fabric 10a to exhibit power for pressing the electrode 20 against the skin when the electrode 20 is mounted to the first clothing fabric 10a.

The first clothing fabric 10a preferably has a larger area than the area of the second clothing fabric 10b. This configuration enlarges the contact area between the clothing fabric around the electrode 20 and the skin and therefore makes the electrode 20 less likely to be displaced when the electrode 20 is mounted to the first clothing fabric 10a.

The first band portion 11 and the second band portion 12 may be, for example, fixed to each other directly or indirectly when the biological information measuring garment for cattle 1 is worn by the cow 100. For example, as illustrated in FIG. 1, the first band portion 11 and the second band portion 12 preferably include a first joining member 31 and a second joining member 32, respectively. Examples of the first joining member 31 and the second joining member 32 include hook and loop fasteners such as MAGICTAPE (registered trade name) and FREEMAGIC (registered trade name), and a buckle. Alternatively, the first joining member 31 and the second joining member 32 may be joined to each other via, for example, a removable belt. This removable belt enables easy adjustment of the length in the body peripheral direction C and therefore enables the biological information measuring garment for cattle 1 to be easily worn. Further, this removable belt makes the electrode 20 less likely to be displaced and improves the measurement accuracy. Examples of the removable belt include a belt including, on both ends thereof, hook and loop fasters such as MAGICTAPE (registered trade name) and FREEMAGIC (registered trade name), and a buckle. As to the material for the first band portion 11, the second band portion 12, and the removable belt, the materials for the first clothing fabric 10a and the second clothing fabric 10b can be referred to. Other examples of the material for the first band portion 11, the second band portion 12, and the removable belt include rubber and leather.

The first band portion 11 and the second band portion 12 preferably extend toward the body peripheral direction C. This configuration enables easy fastening of the biological information measuring garment for cattle 1.

The first band portion 11 and the second band portion 12 may be formed of a clothing fabric made of a different material from the material of the main portion 10c, or may be formed of a clothing fabric made of the same material as the material of the main portion 10c. In FIG. 1, the first band portion 11 and the second band portion 12 are fixed at one end thereof to the main portion 10c by sewing, but may be seamlessly and integrally formed with the main portion 10c. In addition, the first band portion 11 and the second band portion 12 may be formed of the same material or may be formed of different materials.

The first band portion 11 and the second band portion 12 are preferably positioned at an abdomen of the cow 100 when the biological information measuring garment for cattle 1 is worn by the cow 100. This configuration enables easy adjustment of fastening power by the biological information measuring garment for cattle 1.

The electrode 20 is provided on the skin-side surface of the clothing fabric 10. At least a part of the electrode 20 is preferably positioned at a portion on a body side of the cow 100 when the biological information measuring garment for cattle 1 is worn by the cow 100. This configuration enables easy acquisition of electrocardiographic information. Further, the electrode 20 is preferably positioned behind front legs of the cow 100. Positioning the electrode 20 at a portion behind the front legs of the cow 100 can reduce the risk in which the cow 100, for example, bends its neck and takes into its mouth an electronic device provided in the vicinity of the electrode 20 and thus causes the electronic device to fall off or to be damaged.

The electrode 20 is preferably provided in the region (central region R) from the first band portion 11 to the second band portion 12 of the clothing fabric 10. Since the central region R is positioned on an extended line of the first band portion 11 and the second band portion 12 and is a part to which large fastening power is applied, providing the electrode 20 in the central region R can easily prevent the electrode 20 from being positionally displaced.

The clothing fabric 10 includes a portion having the electrode 20 provided therein, the portion preferably having a length L3 in the front-back direction that is shorter than a maximum length L10 in the front-back direction of the clothing fabric 10. This configuration makes the fastening power to be easily concentrated in the vicinity of the electrode 20 and therefore facilitates close attachment of a region in the vicinity of the electrode 20 to the skin of the cow 100. Specifically, the length L3 is preferably 0.95 times or less, more preferably 0.90 times or less, further preferably 0.70 times or less, further more preferably 0.60 times or less the maximum length L10. On the other hand, the lower limit is not particularly limited, and may be 0.3 times or more, 0.4 times or more. When a plurality of electrodes 20 are provided, at least one of portions in which the electrodes 20 are provided preferably satisfies the range described above, and all the portions in which the electrodes 20 are provided more preferably satisfy the range described above.

A portion of the clothing fabric 10 that has the maximum length L10 is preferably positioned at a backbone part of the cow 100. This configuration makes the biological information measuring garment for cattle 1 further less likely to be displaced.

The first band portion 11 has a length L1 in the front-back direction that is preferably shorter than the length L3 in the front-back direction of the portion having the electrode 20 provided therein. This configuration enables easy fastening of the biological information measuring garment for cattle 1. Specifically, the length L1 is preferably 0.7 times or less, more preferably 0.6 times or less, further preferably 0.5 times or less the length L3. On the other hand, the lower limit is not particularly limited, and may be 0.1 times or more, 0.2 times or more. When a plurality of electrodes 20 are present, at least one of portions in which the electrodes 20 are provided preferably satisfies the range described above, and all the portions in which the electrodes 20 are provided more preferably satisfy the range described above.

The second band portion 12 has a length L2 in the front-back direction that is preferably shorter than the length L3 in the front-back direction of the portion having the electrode 20 provided therein. This configuration enables easy fastening of the biological information measuring garment for cattle 1. Specifically, the length L2 is preferably 0.7 times or less, more preferably 0.6 times or less, further preferably 0.5 times or less the length L3. On the other hand, the lower limit is not particularly limited, and may be 0.1 times or more, 0.2 times or more. When a plurality of electrodes 20 are present, at least one of portions in which the electrodes 20 are provided preferably satisfies the range described above, and all the portions in which the electrodes 20 are provided more preferably satisfy the range described above.

The electrode 20 preferably includes an insulating layer formed on the skin-side surface of the clothing fabric 10, and a conductive layer formed on the insulating layer.

The insulating layer may be formed of, for example, an insulating resin, and the type of resin is not particularly limited. As the resin, it is possible to preferably use, for example, a polyurethane-based resin, a silicone-based resin, a vinyl chloride-based resin, an epoxy-based resin, or a polyester elastomer. Among these resins, a polyurethane-based resin is more preferable, and has excellent adhesiveness to the conductive layer. These resins may be used singly, or two or more types thereof may be used. The method for forming the insulating layer is not particularly limited, and the insulating layer can be formed, for example, by dissolving or dispersing the insulating resin in a solvent (preferably water), applying the resulting solution to or performing printing with the resulting solution on release paper or a release film to form a coating film, and volatilizing the solvent included in the coating film and thus drying the coating film. A commercially available resin sheet or resin film can also be used. The insulating layer preferably has an average film thickness of 10 to 200 µm.

The conductive layer is a layer for securing conduction. The conductive layer preferably includes a conductive filler and a resin. The resin is preferably a stretchable resin.

As the stretchable resin, for example, a urethane resin, natural rubber, synthetic rubber, an elastomer, silicone rubber, or fluoro-rubber is preferable, and a stretchable resin including at least rubber containing a sulfur atom and/or rubber containing a nitrile group is more preferable. The sulfur atom and the nitrile group have a high affinity for the conductive filler (particularly a metal powder), and rubber has high stretchability and can therefore easily avoid the generation of cracks or the like at the time of extension. Such a resin enables the conductive filler to be easily retained in a uniformly dispersed manner and can reduce the change ratio of electric resistance at the time of extension. Examples of the rubber containing a sulfur atom include, in addition to rubber containing a sulfur atom, an elastomer containing a sulfur atom. The sulfur atom is contained in the form of a sulfide bond or a disulfide bond in the main chain of a polymer, a mercapto group in a side chain or at a terminal, or the like. Examples of the rubber containing a nitrile group include, in addition to rubber containing a nitrile group, an elastomer containing a nitrile group. Particularly, an acrylonitrile-butadiene copolymer rubber that is a copolymer of butadiene and acrylonitrile is a preferable example. As a commercially available product that can be used as the rubber containing a nitrile group, “Nipol (registered trade name) 1042” manufactured by Zeon Corporation is a preferable example. A single type of resin, or two or more types of resins may be used as the stretchable resin included in the conductive layer. In the entire resin of the conductive layer, the total amount of the rubber containing a sulfur atom and the rubber containing a nitrile group is preferably 95 mass% or more, more preferably 98 mass% or more, further preferably 99 mass% or more.

As the conductive filler, it is possible to use, for example, a metal powder, metal nanoparticles, or a conductive material other than a metal powder. A single type of material, or two or more types of materials may be used as the conductive filler. Examples of the metal powder include noble metal powders such as a silver powder, a gold powder, a platinum powder, and a palladium powder; base metal powders such as a copper powder, a nickel powder, an aluminum powder, and a brass powder; a plated powder obtained by plating different types of particles made of inorganic substances such as a base metal and silica with a noble metal such as silver; and an alloyed base metal powder obtained by alloying a base metal and a noble metal such as silver.

The conductive layer can be formed using, for example, a composition (hereinafter, sometimes referred to as a conductive paste) obtained by dissolving or dispersing components in an organic solvent. The resin in the conductive layer (in other words, the solid content of the stretchable resin in the total solid content of the conductive paste for forming the conductive layer) is preferably 5 to 50 mass%, more preferably 10 to 40 mass%. On the other hand, the conductive filler in the conductive layer is preferably 50 to 95 mass%, more preferably 60 to 90 mass%. This configuration enables the conductive layer to easily attain both conductivity and stretchability.

The conductive layer can be formed directly on the insulating layer, using a composition (conductive paste) obtained by dissolving or dispersing components in an organic solvent, or can be formed by forming a coating film through applying the composition or performing printing with the composition in a desired pattern, and volatilizing the organic solvent included in the coating film and thus drying the coating film. The conductive layer may also be formed, for example, by applying the conductive paste to or performing printing with the conductive paste on a release sheet or the like to form a coating film, volatilizing the organic solvent included in the coating film and thus drying the coating film to form a sheet-shaped conductive layer in advance, and stacking the sheet-shaped conductive layer in a desired pattern on the insulating layer.

The conductive layer has a dried film thickness of preferably 10 to 150 µm, more preferably 20 to 130 µm, further preferably 30 to 100 µm. This configuration enables the conductive layer to attain both durability and wear comfort.

The conductive layer is preferably covered with an insulating layer except for exposed portions such as a skin contact portion (electrode portion) in contact with the skin and a connection portion of a connector. This insulating layer is called a second insulating layer. Providing the second insulating layer can prevent the conductive layer from contacting with water such as rain, snow, and sweat. As to a resin forming the second insulating layer, the description of the resin forming the insulating layer (hereinafter called a first insulating layer) formed on the skin-side surface of the clothing fabric 10 can be referred to. The resin forming the second insulating layer may be the same as or different from the resin forming the first insulating layer, but is preferably the same. The use of the same resin can reduce damage on the conductive layer caused by bias in stress during stretch or contraction of the conductive layer and the insulating layers. The second insulating layer can be formed by the same forming method as the first insulating layer. The second insulating layer preferably has an average film thickness of 10 to 200 µm.

The electrode 20 in FIG. 1 is formed by providing the first insulating layer on the skin-side surface of the clothing fabric 10 and providing the conductive layer on the first insulating layer. Further, the second insulating layer is provided on the conductive layer except for the electrode 20 and an electronic device connection portion 21. In the electronic device connection portion 21, an electronic device can be mounted to the outer side surface of the clothing fabric 10 via a connector such as a snap fastener.

The electrode 20 is formed of a conductive structure, and when the conductive structure has a load of 14.7 N applied thereto in a body length direction or a body width direction, the conductive structure preferably has an extension percentage of 3% or more and 60% or less in at least one of the directions. The conductive structure having an extension percentage of 3% or more allows the electrode 20 to sufficiently and easily follow movement of the clothing fabric 10 and thus makes the electrode 20 less likely to be peeled from the clothing fabric 10. For this reason, the conductive structure has an extension percentage of more preferably 5% or more, further preferably 10% or more. On the other hand, the conductive structure having an extension percentage of 60% or less can easily prevent the measurement accuracy of biological information from being degraded due to excessive stretch of the electrode 20. For this reason, the conductive structure has an extension percentage of more preferably 55% or less, further preferably 50% or less. The extension percentage can be measured, for example, by obtaining a test piece with a prescribed size, mounting the test piece to an Instron tensile tester, and subjecting the test piece to measurement at a speed of 300 mm/min and a load of 14.7 N.

Examples of the electrode having a conductive structure include a woven fabric, a knitted fabric, and a non-woven fabric made of a conductive fiber or a conductive yarn obtained by covering a base fiber with a conductive polymer; a fiber having a surface thereof covered with a conductive metal such as silver, gold, copper, or nickel; a conductive yarn made of a conductive fine metal wire; or a conductive yarn obtained by mixed spinning of a conductive fine metal wire and a non-conductive fiber. Further, one obtained by embroidering a non-conductive fabric with the conductive yarn can also be used as the electrode having a conductive structure.

The biological information measuring garment for cattle 1 is preferably configured to measure electrocardiographic information. The biological information measuring garment for cattle 1 is less likely to cause positional displacement of the electrode 20 and can therefore be suitably used for measuring electrocardiographic information.

The biological information measuring garment for cattle 1 preferably includes an electronic device having a function of calculating electric signals acquired through the electrode. The electronic device can calculate and process electric signals acquired through the electrode. This configuration allows acquisition of biological information such as cardiac potential, a heart rate, a pulse rate, a breathing rate, blood pressure, body temperature, myopotential, and sweating. The electronic device is preferably detachable from the garment. Further, the electronic device preferably includes display means, storage means, communication means, a USB connector, and the like. The electronic device may also include, for example, a sensor capable of measuring environmental information such as atmospheric temperature, humidity, or atmospheric pressure, or a sensor capable of measuring positional information using a GPS.

The biological information measuring garment for cattle 1 can measure cardiac potential, myopotential, and the like, for example, by including two or more electrodes for measuring biological information on the skin-side surface of the biological information measuring garment for cattle. Providing a non-contact electrode on the skin-side surface or the surface of the biological information measuring garment for cattle 1 enables measurement of impedance changes of the body and thus measurement of pulse, breathing, an exercise state, or the like.

The biological information measuring garment for cattle 1 covers preferably at least a part of a thorax, an abdomen, a back, a front leg, a rear leg, a neck, or a face of the cow, more preferably at least a part of a thorax or an abdomen. Examples of the cattle that are made to wear the biological information measuring garment for cattle 1 include livestock cattle and a dairy cow, and a dairy cow is preferable.

The present application claims priority based on Japanese Patent Application No. 2020-011435 filed on Jan. 28, 2020. All the contents described in Japanese Patent Application No. 2020-011435 filed on Jan. 28, 2020 are incorporated herein by reference.

### EXAMPLES

Hereinafter, the present invention is more specifically described by way of examples. The present invention, however, is not limited by the following examples, and can also be implemented with changes to the examples within a scope in compliance with the intent described above and below, and all the changes are to be encompassed within the technical scope of the present invention.

### Example 1

A clothing fabric was prepared that had the same shape as the clothing fabric 10 illustrated in FIG. 1. First, a urethane sponge sheet manufactured by KAZUWA CORPORATION was, as a base material, cut into a substantially trapezoidal shape represented by the first clothing fabric 10a. Specifically, the urethane sponge sheet was cut into a substantially trapezoidal shape represented by the first clothing fabric 10a in FIG. 1 so that it had a maximum length in the front-back direction of 33 cm, a minimum length in the front-back direction of 12 cm, and a length in the body peripheral direction C of 93 cm. Further, both surfaces of the urethane sponge sheet were covered with circular knitted fabrics (ECO-1085) manufactured by Toray Industries, Inc. Next, a W-raschel-mesh fabric (FT6000) manufactured by Toray Industries, Inc. was cut into a trapezoidal shape represented by the second fabric 10b. Specifically, the W-raschel-mesh fabric was cut into a trapezoidal shape with a lower base of 30 cm, an upper base of 9 cm, and a height of 45 cm. Thereafter, these materials were sewn together with piping to form a main portion 10c. Further, an urban twill fabric manufactured by Masuda Co., Ltd. was cut into a size of 6 cm × 140 cm, and the fabric was equipped with FREEMAGIC (38 cm × 130 cm) manufactured by Kuraray Fastening Co., Ltd. and fixed to the main portion 10c by sewing, thus forming a first band portion 11. In addition, an urban twill fabric manufactured by Masuda Co., Ltd. was cut into a size of 6 cm × 10 cm, the fabric was equipped with a metal eight-ring buckle and fixed to the main portion 10c by sewing, thus forming a second band portion 12. Thus, a garment for cattle was obtained.

### Example 2

A main portion 10c of a clothing fabric was formed similarly to Example 1. Further, an urban twill fabric manufactured by Masuda Co., Ltd. was cut into a size of 4 cm × 75 cm, and the fabric had one end thereof equipped with a resin eight-ring buckle and had the other end fixed to the main portion 10c by sewing, thus forming a first band portion 11. Similarly, an urban twill fabric manufactured by Masuda Co., Ltd. was cut into a size of 4 cm × 75 cm, and the fabric had one end thereof equipped with a resin eight-ring buckle and had the other end fixed to the main portion 10c by sewing, thus forming a second band portion 12. Further, separately from these fabrics, an urban twill fabric manufactured by Masuda Co., Ltd. was cut into a size of 4 cm × 140 cm, and the fabric had both ends thereof equipped with resin eight-ring buckles, thus preparing a belt. Next, the resin eight-ring buckles on both ends of the belt were joined with the resin eight-ring buckle of the first band portion 11 and the resin eight-ring buckle of the second band portion 12, and thus a garment for cattle was obtained. The belt was removable.

### Comparative Example 1

A garment for cattle was obtained by the same method as in Example 1 except that the front-back direction of the clothing fabric 10 was reversed, that is, except that the front-side region F of the clothing fabric 10 was formed to have a smaller area than the area of the back-side region B.

An electrode was provided on the skin-side surface of the garments for cattle of Examples 1 and 2 and Comparative Example 1 that were obtained as described above, and thus biological information measuring garments for cattle were produced. The details are as follows.

Twenty parts by mass of nitrile rubber (Nipol DN003 manufactured by Zeon Corporation) was dissolved as a resin in 80 parts by mass of isophorone to prepare an NBR solution. In 100 parts by mass of this NBR solution were blended 110 parts of silver particles (“agglomerate silver powder G-35” manufactured by DOWA Electronics Materials Co., Ltd., average particle size: 5.9 µm), and the mixture was kneaded by a triple roll mill to give a stretchable silver paste.

Next, the stretchable silver paste was applied onto a release sheet and dried by a hot air dry oven set to 120° C. for 30 minutes or more, to prepare a release sheet-attached sheet-shaped conductive layer.

Next, onto a conductive-layer surface of the release sheet-attached sheet-shaped conductive layer, a polyurethane hot-melt sheet was attached, and the release film was then peeled to give a polyurethane hot-melt sheet-attached sheet-shaped conductive layer. The polyurethane hot-melt sheet was stacked using a hot pressing machine under the conditions of a pressure of 0.5 kg/cm², a temperature of 130° C., and a press time of 20 seconds.

Next, the polyurethane hot-melt sheet-attached sheet-shaped conductive layer having a length of 12 cm and a width of 2 cm was stacked on a polyurethane hot-melt sheet having a length of 13 cm and a width of 2.4 cm, with the polyurethane hot-melt sheet side of the conductive layer directed to the polyurethane hot-melt sheet, and a laminate of the polyurethane hot-melt sheet and the sheet-shaped conductive layer was thus produced. The polyurethane hot-melt sheet corresponds to the first insulating layer described above.

Next, the same polyurethane hot-melt sheet as the one used to form the first insulating layer was stacked so as to cover a part of the first insulating layer and the conductive layer, in a region with a length of 5 cm and a width of 2.4 cm, and from a part 2 cm away from an end, and thus a second insulating layer was formed on a part of the conductive layer. That is, a skin contact electrode was produced in which an electronic device connection portion, an insulating portion, and an electrode were longitudinally disposed in this order, the electronic device connection portion exposing the conductive layer on one end of the skin contact electrode and having a size of length 2 cm × width 2 cm, the insulating portion having a stack structure of the first insulating layer/the conductive layer/the second insulating layer, and the electrode exposing the conductive layer on the opposite end and having a size of length 5 cm × width 2 cm.

Next, two skin contact electrodes were attached symmetrically as illustrated in FIG. 1 at prescribed positions on the skin-side surface of the garments for cattle of Examples 1 and 2 and Comparative Example 1. Further, an electrocardiographic measurement device, myBeat manufactured by UNION TOOL CO. was connected to the electronic device connection portion via a snap button as a connector.

### Wearing Test

A dairy female cow was made to wear each of the biological information measuring garments for cattle obtained in Examples 1 and 2 and Comparative Example 1 and was subjected to electrocardiographic measurement in walking for 15 minutes. As a result, the biological information measuring garments for cattle of Examples 1 and 2 in which the clothing fabric had a larger area in the front-side region than an area in the back-side region gave a stable measurement result. On the other hand, the biological information measuring garment for cattle of Comparative Example 1 in which the clothing fabric had a smaller area in the front-side region than an area in the back-side region allowed the electrode to positionally displaced due to walking and did not give a stable measurement result.

### Description of Reference Signs

1 Biological information measuring garment for cattle
10 Clothing fabric
10a First clothing fabric
10b Second clothing fabric
10c Main portion
11 First band portion
12 Second band portion
13a First tapered portion
13b Second tapered portion
20 Electrode
21 Electronic device connection portion
31 First joining member
32 Second joining member
100 Cow
C Body peripheral direction
C1 One end
C2 Other end
F Front-side region
R Central region
B Back-side region
L1 Length in front-back direction of first band portion
L2 Length in front-back direction of second band portion
L3 Length in front-back direction of portion, which has electrode provided therein, of clothing fabric
L10 Maximum length in front-back direction of clothing fabric

1. A biological information measuring garment for cattle, the biological information measuring garment including:
a clothing fabric; and
an electrode provided on a skin-side surface of the clothing fabric,
the clothing fabric including a first band portion on one end and a second band portion on another end in a body peripheral direction of the clothing fabric, and
the clothing fabric having a larger area in a front-side region ahead of a region from the first band portion to the second band portion of the clothing fabric than an area in a back-side region behind the region from the first band portion to the second band portion.

2. The biological information measuring garment according to claim 1, wherein
the electrode is provided in the region from the first band portion to the second band portion of the clothing fabric.

3. The biological information measuring garment according to claim 1, wherein
the clothing fabric includes a portion having the electrode provided therein, the portion having a length in a front-back direction that is shorter than a maximum length in the front-back direction of the clothing fabric.

4. The biological information measuring garment according to claim 1, wherein,
the first band portion has a length in the front-back direction that is shorter than the length in the front-back direction of the portion having the electrode provided therein.

5. The biological information measuring garment according to claim 1, wherein,
the second band portion has a length in the front-back direction that is shorter than the length in the front-back direction of the portion having the electrode provided therein.

6. The biological information measuring garment according to claim 1, wherein
the front-side region of the clothing fabric includes a curved portion including an outer edge curving toward a back direction.

7. The biological information measuring garment according to claim 1, wherein
the electrode includes an insulating layer formed on the skin-side surface of the clothing fabric, and a conductive layer formed on the insulating layer.

8. The biological information measuring garment according to claim 1, wherein
the conductive layer includes a conductive filler and a resin.

9. The biological information measuring garment according to claim 1, wherein
the electrode is formed of a conductive structure, and when the conductive structure has a load of 14.7 N applied thereto in a body length direction or a body width direction, the conductive structure has an extension percentage of 3% or more and 60% or less in at least one of the directions.

10. The biological information measuring garment according claim 1, being configured to measure electrocardiographic information.

11. The biological information measuring garment according to claim 1, being configured to cover at least a part of a thorax, an abdomen, a back, a front leg, a rear leg, a neck, or a face.
